(19) United States
(12) Patent Application Publication
(10) Pub. No.: US 2012/0129953 A1
(43) Pub. Date: May 24, 2012

(54) TINGLING AND SALIVATING COMPOSITIONS

(75) Inventors: Anh Le, Princeton, NJ (US); Catherine Maurel, Plan-les-Ouates (CH)

(73) Assignee: FIRMENICH SA, Geneva (CH)

(21) Appl. No.: 13/362,023

(22) Filed: Jan. 31, 2012

Related U.S. Application Data
(63) Continuation-in-part of application No. 12/300,586, filed on Nov. 12, 2008.

(30) Foreign Application Priority Data
Jun. 12, 2006 (EP) 06115325.0

(51) Int. Cl. A23L 1/222; A23G 4/06; A23L 2/56; A23G 3/50; A23G 3/48; A23K 1/00; A61K 47/26; A23G 3/36

(52) U.S. Cl. 514/777; 426/651; 426/3; 426/660; 426/632; 426/590; 426/2

(57) ABSTRACT

A sensate composition comprises a salivating component, a tingling component and a flavor, the salivating component being formed of at least a mixture of citric, malic and succinic acid, or of their salts, and the tingling component being formed of an ingredient selected from the group consisting of Jambu oleoresin, spilanthol, any extract containing either or both, and their mixtures. The sensate composition allows the preparation of consumer products having improved salivating properties.

### CROSS-REFERENCE TO RELATED APPLICATIONS

This application is a continuation-in-part of application Ser. No. 12/300,586 filed Nov. 12, 2008, which is the 371 filing of International Patent Application PCT/IB2007/052044 filed May 31, 2007. The entire content of each earlier filed application is expressly incorporated herein by reference thereto.

### TECHNICAL FIELD

The present invention relates to the field of food ingredients and flavor compositions capable of creating useful sensations in the mouth upon food consumption. In particular, the present invention relates to a salivating and tingling composition comprising a salivating agent which contains an acidulent base, and a tingling ingredient. Preferably the tingling ingredient is in encapsulated form, namely in the form of an extruded product, and contains Jambu oleoresin.

The invention also relates to the use of this composition in orally ingestible or masticated consumer products, in particular confectionery products such as hard-boiled candy, chewing gum and compressed tablets, but also generally in foods and beverages, dairy products, savory products such as snacks and ready-to-eat meals, pet foods, functional foods such as nutrition supplement type products, traditional oral care products such as toothpaste, mouthwashes and breath films, and pharmaceuticals.

### BACKGROUND OF THE INVENTION

The particular sensations mediated by the trigeminal nerve, such as cooling, warming, pungent and salivating, stimulate consumers' senses upon consumption of the orally ingestible products capable of providing this type of effect. The present invention seeks to provide compositions that combine the capacity to overcome dry mouth or xerostomia problems with the delivery of a tingling/electric type sensation in the mouth, which is especially appreciated of consumers in beverages and confectionery products in particular.

There have been reports in the prior art of compositions able to remedy xerostomia problems. For example, EP 1 454 533 A1, to International Flavors & Fragrances Inc., discloses products that promote saliva generation in the mouth, through the use of various food grade acceptable acids, in foods such as chewing gums, breath films, toothpaste and lozenges. It discloses in particular food grade compositions comprising a flavoring component, the flavoring component containing at least about 8 weight percent acid or acid salts selected from the group consisting of citric, malic, adipic, tartaric, glutaric and fumaric acids; wherein the food grade composition spends at least one minute in the mouth.

US patent application 20030118628 A1, which also addresses the issues related to the xerostomia conditions and the need to provide products capable of alleviating them, discloses confectionery products comprising a salivation agent and an oral comfort ingredient which is separate and distinct from the region where the salivating agent is located in the confectionery in the confectionery product. The confectionery base may be a carbohydrate or carbohydrate derivative, namely a sugar, and the salivation agents include acidulants, cooling compounds, salts, salt enhancers, monosodium glutamate (MSG), MSG enhancers, flavors and mixtures thereof. Mixtures of acidulants include citric acid, malic acid, succinic acid and adipic acid.

Other US documents, U.S. Pat. Nos. 4,820,506 and 4,151,270, relate to the use of liquids for the promotion of human saliva, containing organic acidulants, namely citric, malic or ascorbic acid, in a synergistic mixture with a sweetener such as aspartame or saccharin. In a similar context, U.S. Pat. No. 4,088,788 also discloses synergetic mixtures of organic acids with saccharin and sodium and potassium salts to stimulate salivation provided by chewing gums, whilst reducing muscular fatigue. US 2006/0204551-A1 discloses a salivation cocktail that comprises a food acid and a tingling sensate and mentions various organic acids among a long list of candidates for use as the food acid component.

More recent U.S. Pat. Nos. 5,057,328 and 6,770,308 describe the use of acidulants in an encapsulated form in a polyvinyl acetate matrix. It is disclosed that the encapsulated acidulants are less degraded when undergoing extrusion cooking, thus providing longer lasting tartness and flavor to the confectionery products in which they are incorporated. In a similar context, U.S. Pat. No. 6,399,141 teaches the preparation of granular food acid compositions obtained by spray granulation methods and containing fumaric acid.

All of the above mentioned prior art documents attest to the general usefulness of acid mixtures to stimulate salivation, of attempts to obtain synergies of the latter with sweeteners, and of certain developments aimed at improving the stability of these food acid mixtures during the cooking processes used in the manufacture of many cooked confectionery products. None of the above documents addresses or even suggests the object of the present invention.

The prior art is also rich in disclosures of sensate mixtures capable of stimulating the trigeminal nerve to provide cooling, warming and tingling sensations. One can cite in this context, among the many prior art reports in this field, the recent U.S. Pat. Nos. 6,899,901 and 6,780,443. These documents relate to the use of sensate compositions comprising cooling, warming and tingling components, the tingling component comprising Jambu oleoresin or spilanthol. These documents do not address the object of the present invention, nor do they contain any teaching with regard to salivating mixtures.

In conclusion, despite the abundance of prior art disclosures of compositions capable of providing stimulation of the trigeminal nerve to induce sensations of the cooling, warming, tingling and salivating type in the animal and human mouth, to the best of our knowledge there has never been any disclosure or suggestion of a combination of ingredients such as that presently claimed to achieve a synergistic effect between mixtures of well-known acidulants with a tingling ingredient of Jambu oleoresin or spilanthol.

We have now surprisingly established that the sensate compositions according to the present invention make it possible to enhance the salivating effect of the prior known salivation agents based on mixtures of food organic acids such as citric, malic, succinic and similar acids, whilst delivering a much desired tingling sensation via the use of certains forms of Jambu oleoresin or spilanthol. As will become apparent from the disclosure hereafter, the use of extruded forms of the latter in particular, together with certain combinations of solid and liquid acidulant ingredients, deliver surprising compositions able to provide unexpected salivating effects, in particular in confectionery consumer products.

### SUMMARY OF THE INVENTION

The present inventors have established that by preparing a composition comprising a salivating component, a flavor and a tingling component, wherein the salivating component comprises at least a mixture of citric, malic and succinic acids or precursors thereof, and the tingling component is formed of an ingredient selected from the group consisting of Jambu oleoresin, spilanthol, extracts containing either or both, and their mixtures, sensate compositions delivering improved salivating effects could be obtained.

Accordingly, the present invention provides, in a first aspect, a sensate composition having the above characteristics.

In a second aspect, the present invention provides the use of a sensate composition as described above to prepare consumer products that are intended for oral ingestion, to be masticated or to be used to wash, freshen or sanitize buccal cavities and more particularly the human mouth.

In further aspects, the invention provides consumer products such as cited here-above comprising the sensate composition of the invention, in particular a food product, an oral care product, a pharmaceutical product or nutritional supplement.

Other embodiments of the invention relate to a method for stimulating salivation, which comprises consuming or using a consumer product as recited here above.

It is a further objective of the invention to provide sensate compositions and consumer products containing the latter, which promote salivation and are substantially devoid of any off-notes commonly associated with the use of tingling ingredients in particular, namely the Jambu oleoresin and the natural extracts containing the latter, in which high concentrations of these compounds are present, such that the gustative properties of the consumer product are not adversely affected by the presence of this tingling ingredient.

### DESCRIPTION OF THE FIGURES

FIG. 1 shows a comparison of the perceived salivating sensation by trained panellists from hard-boiled candies prepared according to the invention and compared to prior art controls (Example 1), as a result of blind test panel evaluation. Differences are statistically significant at the 95% (*) and 99% (**) level of confidence.

FIG. 2 shows a comparison of the perceived salivating sensation by trained panellists from chewing gum prepared according to the invention and compared to prior art controls (Example 2), as a result of blind test panel evaluation. Differences are statistically significant at the 95% (*) and 99% (**) level of confidence.

FIG. 3 shows a comparison of the perceived salivating sensation by trained panellists from compressed tablets prepared according to the invention and compared to prior art controls, as a result of blind test panel evaluation. Differences are statistically significant at the 95% (*) and 99% (**) level of confidence.

### DETAILED DESCRIPTION OF THE INVENTION

The sensate composition of the present invention comprises a salivating component and a tingling component and a flavor, the salivating component being formed of at least a mixture of citric, malic and succinic acid, or of their salts, and the tingling component being formed of an ingredient selected from the group consisting of Jambu oleoresin, spilanthol, any extract containing either or both, and their mixtures.

Following a preferred embodiment, the composition of the invention further comprises glycyrrhizin, preferably in an ammoniated form.

According to useful embodiments, the salivating component of the compositions of the invention is formed of two components, a solid mixture of food organic acids and a liquid salivating base which may contain the flavor or flavoring components. Both these parts contain food organic acids commonly used as food acidulants, as cited in the representative prior art discussed before. The salivating component contains at least a mixture of citric, malic and succinic acids, or their salts. It may further comprise other commonly used food acidulants, in particular phosphoric, glutaric, adipic, lactic or yet fumaric acids. It is also clear that the food acceptable salts of such acids are convenient salivating ingredients according to the invention.

The total concentration of salivating component in the sensate composition of the invention is preferably comprised between 10 and 98 weight %, relative to the total weight of the sensate composition, the tingling component forming typically 0.005 to 1% by weight of the mixture, preferably from 0.01 to 0.1 weight %, and the flavor being also used in an amount comprised between 0.01 and 1% weight of the total sensate composition.

Typically, the sensate compositions of the invention comprises from 0.01 to 60% weight of citric acid, from 0.01 to 50% weight of malic acid and from 0.01 to 30% weight of succinic acid, all weights being relative to the total weight of sensate composition.

As mentioned above, it is preferred to add a certain amount of glycyrrhizin to the sensate composition of the invention to help promote salivation. Preferred concentrations of this material are comprised between 0.1 and 2% by weight, relative to the total weight of the salivating component of the sensate composition.

Preferably, the sensate composition consists essentially of the salivating component, tingling component, glycyrrhizin and a flavor, with the salivating component comprising a mixture of citric, malic and succinic acids or of their salts, the mixture including 1 to 20% citric acid, 1 to 10% malic acid and 1 to 15% succinic acid. The flavor is typically present in an amount of 25 to 75% by weight, and the salivating component represents between 60 to 85% by weight of the sensate composition.

More specifically, the sensate composition consists essentially of 2 to 16% by weight of citric acid, 1 to 5% by weight of malic acid and 2 to 10% by weight of succinic acid, or even 4 to 14% by weight of citric acid, 1 to 4% by weight of malic acid and 3 to 10% by weight of succinic acid.

The tingling ingredient of the sensate compositions is formed of a Jambu oleoresin compound, optionally together with any natural extract containing spilanthol in a concentration of at least 30% weight of the latter, relative to the total weight of the extract, and more preferably 40% or more.

Although it is possible to use the tingling component in any physical form of Jambu oleoresin generally available commercially, it is preferred according to the invention to use the latter in the form of a solid product prepared by common extrusion processes, i.e. encapsulated in a glass matrix such as are described for example in prior art documents U.S. Pat. No. 6,607,771, U.S. Pat. No. 6,607,778, U.S. Pat. No.6,932,982 or yet WO 03/56938.

Typically these are products obtained via extrusion of a carbohydrate melt in which the Jambu oleoresin has been incorporated. Such extrusion methods typically comprise preparing a mixture of a continuous phase carrier containing the component to be encapsulated therein and having a low water content so as to ensure that the glass transition temperature of said mixture is the glass transition temperature of the final product; heating said mixture within a screw extruder to a temperature comprised between 90 and 130° C. to form a molten mass; and extruding the molten mass through a die. The molten mass can then be chopped directly as it exits the die, i.e. at the temperature of extrusion, or be cooled before chopping, to form particles of the desired dimension.

Extrusion processes of this type have been generally described in the prior art, including that cited above, and also particularly in relation to encapsulation of labile flavor and fragrance materials, and a more detailed description of such methods is not warranted here. The process conditions for the manufacturing of the extruded Jambu oleoresin compounds forming part of the sensate composition of the invention can be any of the generally known melt extrusion methods described typically in prior art documents cited above or yet such as in WO 2004/082393 and WO 2006/038067, both to Firmenich S. A. These prior disclosures also describe in detail the nature of the carriers preferred for the preparation of such products.

Typical extruded products convenient for the tingling component of the sensate composition are similar to those available from Firmenich SA, Geneva, Switzerland, and commercialised under the trade names DURAROME® and FLEXAROME®.

Preferably, maltodextrine having DE comprised between 10 and 18, and mixtures thereof with hydrogenated starch hydrolysates, shall be used as the matrix carriers of the extruded tingling component according to the invention.

Such extruded products shall typically comprise concentrations in tingling compound between 1 and 5 weight %, relative to the weight of solid extrudate.

The concentration of tingling component in the composition of the invention varies in a wide range of values, depending on the tingling effect desired and the end consumer product to which the sensate composition is destined. One can cite by way of example, concentrations of tingling component comprised between 0.1 and 1% weight, relative to the total weight of the sensate composition.

As cited earlier, the tingling component may also comprise spilanthol, the active principle of the Spilanthes sp., commonly known as pera cress.

The salivating and tingling components of the sensate compositions according to the invention are present in the latter in relative concentration ratios comprised between 1:1 and 10:1 respectively.

As mentioned above, the compositions may also comprise other ingredients, in particular flavoring ingredients. The latter may be added to the salivating or tingling components of the composition. According to some embodiments of the invention, they are easily incorporated into the salivating components, in the solid ingredient thereof or in a salivating liquid base, or in both, depending on the physical form of the flavor or flavoring composition.

By “flavor or flavoring composition”, it is meant here a flavoring ingredient or a mixture of flavouring ingredients, solvents or adjuvants of current use for the preparation of a flavouring formulation, i.e. a particular mixture of ingredients which is intended to be added to an edible composition or chewable product to impart, improve or modify its organoleptic properties, in particular its flavour and/or taste. Flavouring ingredients are well known to a person skilled in the art and their nature does not warrant a detailed description here, which in any case would not be exhaustive, the skilled flavorist being able to select them on the basis of his general knowledge and according to the intended use or application and the organoleptic effect it is desired to achieve. Many of these flavouring ingredients are listed in reference texts such as in the book by S. Arctander, Perfume and Flavour Chemicals, 1969, Montclair, N.J., USA, or its more recent versions, or in other works of similar nature such as Fenaroli's Handbook of Flavour Ingredients, 1975, CRC Press or Synthetic Food Adjuncts, 1947, by M. B. Jacobs, van Nostrand Co., Inc. Solvents and adjuvants of current use for the preparation of a flavouring formulation are also well known in the art. They allow flavouring formulations to meet technical requirements, such as stability or tonality persistence. The solvent is most of the time part of a flavouring composition. Solvents currently used in this context include for instance benzyl alcohol, propylene glycol, triacetine, vegetable oils, ethanol or limonene. The adjuvants, on the other hand, can have many various functions in a flavouring composition. They include for instance stabilizers. Today, the range of products types and product formulations that are flavoured has become so extensive and subjected to frequent changes that an approach made on a product-by-product basis and on the definition for each case of the adjuvants that can be used is impractical. This is why a list of adjuvants currently used in flavouring formulations is not given here. However, a skilled person in the art, namely a flavorist, is capable of choosing these ingredients as a function of the product to be flavoured and of the nature of the flavoring ingredients contained in the formulation.

The sensate compositions of the invention can be used to modify the organoleptic properties of edible or chewable end-products belonging to food segments as varied as confectionery, beverages or savoury, as well of oral care, pharmaceutical and nutrition supplement type products. More particularly, the composition of the invention can be used in products such as chewing gums, sugar almonds, compressed tablets, hard-boiled candies, chewing candies, and the like, to mention the more current ones in the confectionery field. In the case of beverages, they are suitable to impart tingling and salivating effects to carbonated and non-carbonated beverages, alcoholic beverages and the like. They are also useful for dairy applications such as milk drinks, acidic products such as fruit and milk flavoured drinks, yoghurts and the like.

The compositions of the invention are also suitable to impart tingling and salivating sensations in savoury applications, such as dry-blended seasonings, spices, soups, cheeses, butter spreads and the like.

The compositions of the invention are also advantageously used to improve the salivating sensation of oral care products such as toothpastes, mouthwashes and breath improving films.

The proportions in which the compositions according to the invention can be incorporated into the aforementioned consumer products vary within a wide range of values. These values depend on the nature of the ready-to-consume or chewable product and the nature of the sensate composition used in said product, as well as on the intensity of the functional effect it is desired to achieve.

Typically, the sensate composition of the invention will be added to the end products in an amount sufficient to provide the latter with a concentration in acidulents, i.e. the salivating mixture of food acids, comprised between 0.1 and 10 weight %, more preferably 0.1 and 5%, and a concentration in tingling ingredient comprised between 0.1 and 2 weight %, more preferably 0.1 to 1 weight %, of the total weight of consumer product in which they are incorporated, i.e. of the end product.

Typical flavor concentrations vary from 0.001 to 5% by weight of end product and are more preferably below 2% be weight of the total end product weight.

It goes without saying that the sensate compositions of the invention may be combined with many other active ingredients commonly used in such consumer products. This is the case for example when the sensate compositions are intended to be added to functional foods, such as nutritional products or health supplements, which often contain vitamins, useful fatty acids and the like.

In a general manner, the sensate compositions of the invention improve the tingling and salivating properties of bread, wafers, cookies, crackers, pretzels, pizza, rolls, ready-to-eat breakfast cereals, hot cereals, pasta products, snacks such as fruit snacks, salty snacks, grain-based snacks, and microwave popcorn, dairy products such as yoghurt, cheese, and ice cream, sweet goods such as hard candy, soft candy, and chocolate, beverages, animal feed, pet foods such as dog food and cat food, aqua-culture foods such as fish food and shrimp feed, special purpose foods such as baby food, infant formulas, hospital food, medical foods and pharmaceuticals, sports food, performance food or nutritional bars, fortified foods, food pre-blends or mixes for home or food service use, such as pre blends for soups or gravy, dessert mixes, dinner mixes, baking mixes such as bread mixes, and cake mixes, and baking flour.

The invention also relates to such end consumer products and to methods of improving their organoleptic properties by adding thereto the sensate compositions mentioned above. The blends of acids in the family of citric, malic, succinic, tartaric, phosphoric, aspartic, fumaric, lactic, adipic, acetic acids, or their salt combined with the tingling component described previously, and optionally with glycyrrhizine and flavorings, mint, anis, citrus flavours, or yet any fruit flavor tonalities, which constitute particularly advantageous sensate compositions of the invention, thus provide choice products to be incorporated in a very large variety of consumer products and the examples presented below illustrate specific embodiments thereof which are not to be interpreted as limiting the scope of the invention.

The invention also relates to a method of increasing salivation which comprises administering to a subject one of the consumer products disclosed herein with the consumer product including a saliva increasing amount of the sensate composition therein. In this method, the sensate composition is present in the product in an amount of 0.01 to 10% by weight. Alternatively, the method comprises administering to a subject a saliva increasing amount of a sensate composition disclosed herein, with sensate composition administered in an amount of 0.01 to 10% by weight.

As another aspect of the invention, there is also provided a preferred method of preparing such a consumer product, comprising the steps of:
(i) mixing a solid mixture of citric, malic and succinic acids, or their salts, with a salivating liquid base, to form a salivating component;
(ii) adding to said salivating component a tingling component formed of an ingredient selected from the group consisting of Jambu oleoresin, spilanthol, any extract containing either or both, and their mixtures;
(iii) forming said consumer product according to generally known methods.

As will become apparent from the sensory tests disclosed in some of the following examples, the combination of the tingling ingredient, namely the extruded solids of Jambu oleoresin and/or spilanthol, with the acidulant mixture as claimed, have been ascertained to synergistically improve the salivating effect of the latter and thus make it possible to enhance and improve the salivating effect provided by such consumer products, to alleviate the problems associated with a dry mouth or xerostomic conditions.

The invention will now be illustrated by way of the following examples but is not limited to these examples. Temperatures are given in degrees centigrade and abbreviations have the meaning common in the art.

### EXAMPLES

### Example 1

Preparation of Sugar-Free Hard-Boiled Candies

A liquid salivating component A according to the invention was obtained with the following ingredients:

| Ingredients | Origin | Weight % |
|---|---|---|
| Citric acid | Aldrich, | 1 to 10 |
| | CAS N^{o} 0000077929 | |
| Malic acid | Penta, | 1 to 10 |
| | CAS N^{o} 0006915157 | |
| Succinic acid | Aldrich, | 1 to 10 |
| | CAS N^{o} 0000110156 | |
| Glycyrrhizin, ammoniated | Penta, | 1 to 10 |
| | CAS N^{o} 0053956040 | |
| Flavor with mint; anis; citrus; or | Firmenich SA, Geneva | 30 to 60 |
| fruit tonality | | |

The composition above was then used to prepare sugar-free hard-boiled candies, as follows: Isomalt (100 g) and water (30 g) were mixed and heated to 160° C. in a copper pan. At 160° C., the copper pan was removed from the heat and placed in a warm water bath (40° C.). When the temperature reached 135° C., aspartame, acesulfame-K, the liquid acidulant mixture (1.0%) and flavor were added. The cooked mass was poured at ambient temperature into appropriate Teflon® molds.

The following Table I summarizes the ingredients and respective dosages used in the preparation as described here above of a control hard-boiled candy according to the prior art and containing only citric acid as acidulant and a candy prepared according to the invention.

TABLE I
Hard-boiled Candies and their Components

| | | % by weight in |
|---|---|---|
| Sample | Sensate Composition | finished candy |
| Control | Orange flavor | 0.06 |
| | Citric Acid | 1.00 |
| Invention | Orange flavor | 0.06 |
| Candy | Solid salivating component¹⁾ | 1.00 |
| | Salivating component A | 0.20 |
| | Encapsulated Jambu oleoresin | 0.20 |

¹⁾Solid Mixture of citric, malic and succinic acids

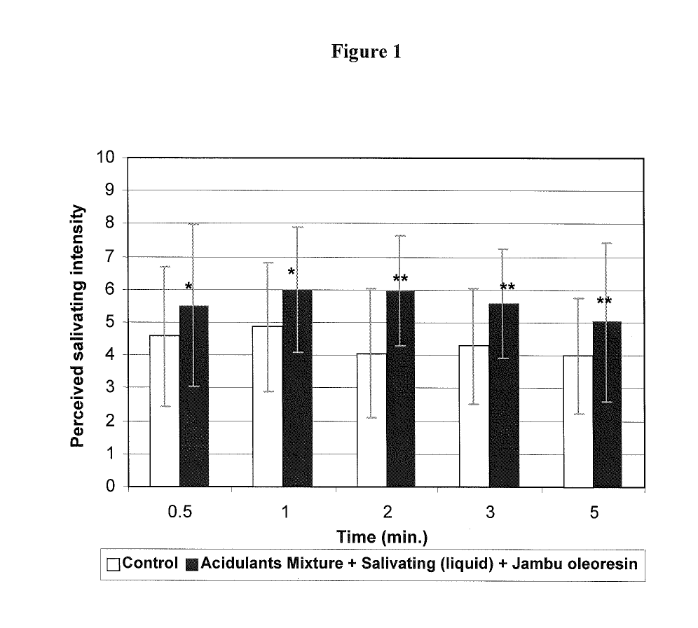

The Control candy and the Invention Candy were then evaluated by a panel of trained evaluators, on a blind test, and the results of this evaluation are shown in FIG. 1.

The sensory results represented in this figure clearly demonstrate the synergistic effect of the presence of the tingling component of encapsulated Jambu oleoresin on the salivating sensation. There was immediate impact on the salivating sensation perceived at 30 seconds and up to 5 minutes after having started the chewing of the candy.

Citric acid, which is commonly used for fruit flavors because of its delivery of impact and sourness, was found to deliver the expected effect. The invention's sensate mixture however was found to provide improved impact and sourness, notably over a significantly longer period. It also delivered a stronger salivating effect than the citric acid alone.

### Example 2

Preparation of Sugar-Free Chewing Gum

Flavored chewing gums were prepared as follows:

crystalline sorbitol, acesulfame K and aspartame were blended in a Turbula blender. Half the blend was mixed with pre-warmed Balear® T and Mistral® gum bases (origin: Cafosa) in a Winkworth sigma-blade mixer at 45-50° C. for 4 minutes. The remaining powder blend was then added along with a humectant syrup (Lycasin® 80/55, glycerin) and mixed for a further 6 minutes. Finally, the flavoring system was added.

The following Table II summarizes the ingredients and respective dosages used in the preparation as described here above of a control chewing gum comprising only a flavor and a chewing gum according to the invention.

TABLE II
Chewing Gums and their Components

| | | % by weight in |
|---|---|---|
| Sample | Sensate Composition | End Product |
| Control | Peppermint flavor⁽¹⁾ | 1.00 |
| Invention | Peppermint flavor⁽¹⁾ | 1.00 |
| Chewing | Salivating Composition A⁽²⁾ | 1.00 |
| Gum | Flexarome ®¹⁾ Jambu oleoresin | 0.40 |

⁽¹⁾origin: Firmenich SA
⁽²⁾Example 1

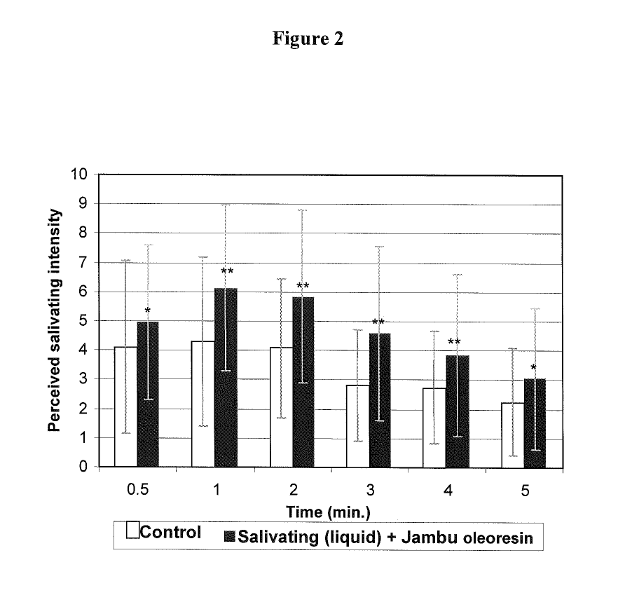

The Control chewing gum and the Invention Chewing Gum were then evaluated by a panel of trained panelists, on a blind test, and the results of this evaluation are shown in FIG. 2. The sensory results represented in this figure clearly demonstrate the synergistic effect of the presence of the tingling component on the salivating sensation. There was immediate impact on the salivating sensation perceived at 30 seconds and up to 5 minutes after having started the chewing.

### Example 3

Preparation of Compressed Tablets

Flavored compressed tablets were prepared as follows:

crystalline sorbitol (97.9%), aspartame (0.05%), acesulfame-K (0.05%) and magnesium stearate (2%) were mixed for 5 minutes in a Turbula (WAB AG, Basel, Switzerland). The flavoring system was added. Tablets, 1.20 g weight, 20 mm diameter, 255 Newton of hardness, were prepared using the Korsch apparatus (Korsch, Germany).

The following Table III summarizes the ingredients and respective dosages used in the preparation as described here above of a control compressed tablet comprising only a flavor and citric acid and a compressed tablet according to the invention.

TABLE III
Compressed Tablets and their Components

| | | % by weight in |
|---|---|---|
| Sample | Sensate Composition | End Product |
| Control | Strawberry Durarome ® flavor⁽¹⁾ | 1.85 |
| | Citric acid | 1.50 |
| Invention | Strawberry Durarome ® flavor⁽¹⁾ | 1.85 |
| Compressed | Spray dried Salivating | |
| Tablet | Composition A⁽²⁾ | 1.50 |
| | Solid salivating component⁽³⁾ | 1.00 |
| | Encapsulated Jambu oleoresin | 0.60 |

⁽¹⁾origin: Firmenich SA
⁽²⁾⁽³⁾Example 1

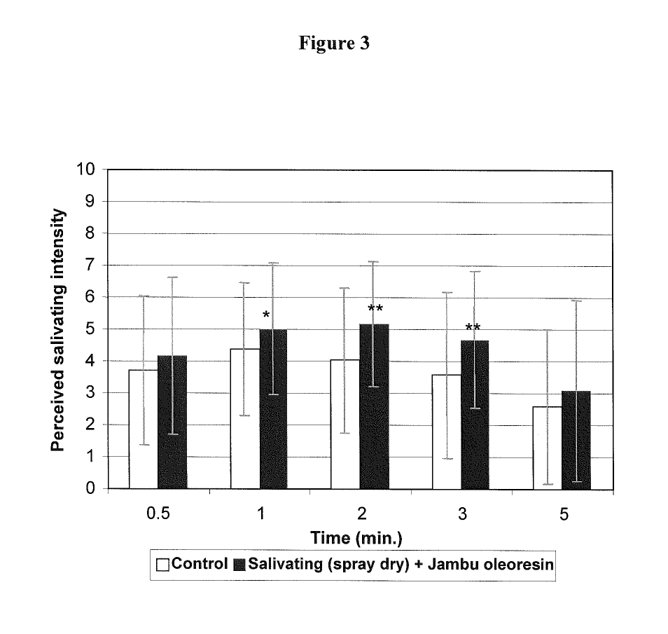

The Control compressed tablet and the Invention Compressed Tablet were then evaluated by a panel of trained panelists, on a blind test, and the results of this evaluation are shown in FIG. 3.

The sensory results represented on this figure clearly demonstrate the synergistic effect of the presence of the tingling component on the salivating sensation. There was immediate impact on the salivating sensation perceived at 30 seconds and up to 5 minutes after having started the chewing.

The same advantages of the invention over citric acid as described in example 1 above were observed.

### Examples 4-6

Preparation of Consumer Products

Similar consumer products as those described in Examples 1 to 3 were prepared by replacing liquid salivating component A with a liquid salivating composition B according to the invention prepared with the following preferred ingredients:

| Ingredients | Origin | Weight % |
|---|---|---|
| Citric acid | Aldrich, | 4 to 20 |
| | CAS N^{o} 0000077929 | |
| Malic acid | Penta, | 3 to 15 |
| | CAS N^{o} 0006915157 | |
| Succinic acid | Aldrich, | 2 to 15 |
| | CAS N^{o} 0000110156 | |
| Phosphoric acid | Penta, | 15 to 30 |
| | CAS N^{o} 0007664382 | |
| Glycyrrhizin, ammoniated | Penta, | 1 to 10 |
| | CAS N^{o} 0053956040 | |
| Flavor with mint; anis; citrus; or | Firmenich SA, Geneva | 30 to 60 |
| fruit tonality | | |
| Fruit juice concentrate or extract | Naturex**: site d'Agroparc | 1 to 10 |
| | BP 1218 | |
| | F-84911 Avignon Cedex 9 | |

Upon blind testing and evaluation of the consumer products thus obtained, carried out in the same conditions as in Examples 1 to 3, similar salivating effect improvements were observed.

### Example 7

Preparation of Toothpaste (Opaque Type)

5 kg of toothpaste were prepared following the protocol and ingredients given below:

Part 1: Solution of Carboxy Methyl Cellulose

| | Amount | Weight |
|---|---|---|
| Ingredients | grams | % |
| Carboxy Methyl Cellulose 7MXF = CMC | 60.00 | 5.66 |
| (Supplier: Hercules BV Aqualon Div.) | | |
| Glycerin | 1000.00 | 94.34 |
| Total | 1060.00 | 100.00 |

Procedure:

Mix CMC and glycerin, using a laboratory mixer at high speed for 5-10 minutes. Let the solution hydrate for 30 minutes

Part 2: Solution of powder ingredients

| | Amount | |
|---|---|---|
| Ingredients | grams | Percent |
| Distilled Water | 1500.00 | 50.74 |
| Saccharin Sodium | 10.00 | 0.34 |
| Sodium Benzoate | 5.00 | 0.17 |
| Sodium Fluoride | 16.00 | 0.54 |
| (Supplier: Riedel de Haen AG - Germany) | | |
| Polyethylene Glycol 1500 | 150.00 | 5.07 |
| Titanium Dioxide | 25.00 | 0.85 |
| Sorbitol 70 | 1250.00 | 42.29 |
| (Supplier: Neosorb 70/70 Roquette Freres) | | |
| Total | 2956.00 | 100.00 |

Procedure:

Pour the dry ingredients (as listed) into water using mixer Turrax for 5-10 minutes. Add Sorbitol liquid, mix 3 minutes. Add Part 1 in Part 2, and mix for 10-15 minutes.

Part 3: Solution of Sodium Lauryl Sulfate

| | Amount | |
|---|---|---|
| Ingredients | grams | Percent |
| Sodium Lauryl Sulphate | 75.00 | 6.38 |
| (Texapon K12 Supplier: Cognis Gmbh - Germany) | | |
| Distilled Water | 200.00 | 17.02 |
| Sorbosil TC 15 | 600.00 | 51.06 |
| (Supplier: Crosfield Limited) | | |
| Sorbosil AC 77 | 300.00 | 25.53 |
| (Supplier: Crosfield Limited) | | |
| Total | 1175.00 | 100.00 |

Procedure:

Mix gently Sodium lauryl sulfate with water and let set for minimum 3 hours, better overnight. Pour the mix of Part 1 and Part 2 into the pan of Stephan Mixer and let set for 2-3 hours, or until the air bubbles disappear. Add Sorbosil (TC 15 and AC 77) and mix under vacuum (50-60 mBar) for 30 minutes. Open the pan and add solution of Sodium lauryl sulfate. Mix under vacuum (50-60 mBar) for 5 minutes.

At the end, flavors were added to the toothpaste. Accordingly, 1% by weight of liquid minty flavour (Firmenich SA, Geneva) and 0.20% by weight of liquid salivating composition B (Examples 4-6) according to the invention were added to the toothpaste thus obtained. The latter provided a salivating, fizzy, bubbling sensation and an improved body and volume sensation during teeth brushing.

### Example 8

Preparation of Mouthwashes

1000 grams of a mouthwash were prepared following the protocol and ingredients described hereafter:

Part 1

| | Amount | |
|---|---|---|
| Ingredients | grams | Percent |
| Water (demineralised) | 765.500 | 76.70 |
| Sodium Monofuorilphosphate | 0.500 | 0.05 |
| (Supplier: Riedel-de-Haen) | | |
| Sodium Saccharine Q.S. | q.s. | q.s. |
| Sodium Benzoate | 2.000 | 0.20 |
| Sorbitol (70%) | 80.000 | 8.02 |
| (Supplier: Neosorb 70/70 Roquette) | | |
| Glycerine | 80.000 | 8.02 |
| Ethanol (95%) | 60.00 | 6.01 |
| Total | 988.000 | 98.80 |

Procedure:

Under magnetic stirring add SMFP, sodium saccharine and sodium benzoate in water without heating.

Add sorbitol, glycerine and ethanol. Let mix 10 minutes or until well dissolved. To be used in undiluted form as a Mouthwash

Part 2

| | Amount | |
|---|---|---|
| Ingredients | grams | Percent |
| Peg 40 - Hydrogenated Castor Oil | 10.000 | 1.00 |
| Comment: Supplier: BASF | | |
| (Cremophor CO 40 - 936037) | | |
| Peppermint Flavor | 2.000 | 0.20 |
| Total | 12.000 | 1.20 |

Procedure:

Mix PEG 40 with flavor. Heat and mix until well dissolved.

Mix Part 1 and Part 2.

TABLE IV
Dosages of flavor and Salivating component in mouthwash

| | | Dosage |
|---|---|---|
| Sample | Flavor | (w/w) |
| Control | Citrus Flavor¹⁾ | 0.12% |
| Test | Citrus Flavor¹⁾ | 0.06% |
| | Salivating Component B | 0.060% |

¹⁾Origin: Firmenich SA, Geneva, Switzerland

TABLE V
Dosages of flavor and Salivating component in mouthwash

| | | Dosage |
|---|---|---|
| Sample | Flavor | (w/w) |
| Control | Peppermint Flavor¹⁾ | 0.20% |
| Test | Peppermint Flavor¹⁾ | 0.10% |
| | Salivating Component B | 0.10% |

¹⁾Origin: Firmenich SA, Geneva, Switzerland

What is claimed is:

1. A sensate composition comprising a salivating component, a tingling component, glycyrrhizin in an amount of 0.1 to 10% to help promote salivation and a flavor, the salivating component comprising a mixture of citric, malic and succinic acids or of their salts, the mixture including 1 to 20% citric acid, 1 to 10% malic acid and 1 to 15% succinic acid, and the tingling component being formed of an ingredient selected from the group consisting of Jambu oleoresin, spilanthol, any extract containing either or both, and their mixtures and being present at a concentration of between 0.1 to 1% by weight, all weights being relative to the total weight of composition.

2. The composition according to claim 1, wherein the flavor is present in an amount of 25 to 75% by weight, and the salivating component represents between 60 to 85% by weight of the sensate composition.

3. The composition according to claim 1, wherein the tingling component is an extruded solid of Jambu oleoresin.

4. The composition according to claim 1, wherein the sensate composition consists essentially of 2 to 16% by weight of citric acid, 1 to 5% by weight of malic acid and 2 to 10% by weight of succinic acid.

5. The composition according to claim 1, wherein the glycyrrhizin is ammoniated glycyrrhizin and is present in an amount of 0.1 to 2% by weight.

6. The composition according to claim 5, wherein the sensate composition consists essentially of 4 to 14% by weight of citric acid, 1 to 4% by weight of malic acid and 3 to 10% by weight of succinic acid.

7. A sensate composition comprising a salivating component, a tingling component, ammoniated glycyrrhizin in an amount of 0.1 to 2% to help promote salivation and a flavor, the salivating component comprising a mixture of citric, malic and succinic acids or of their salts, the mixture including 4 to 14% citric acid, 1 to 4% malic acid and 3 to 10% succinic acid, and the tingling component being formed of an ingredient selected from the group consisting of Jambu oleoresin, spilanthol, any extract containing either or both, and their mixtures and being present at a concentration of between 0.1 to 1% by weight, all weights being relative to the total weight of the sensate composition.

8. A consumer product capable of being ingested orally, masticated or used to clean or sanitize buccal cavities, comprising a sensate composition according to claim 1.

9. The consumer product according to claim 8, comprising 0.01 to 10% by weight of the sensate composition.

10. The consumer product according to claim 9, in the form of a confectionery product such as a chewing gum, sugar almond, compressed tablet or hard-boiled candy.

11. The consumer product according to claim 9, in the form of a pet food, a pharmaceutical composition or a beverage.

12. A method of increasing salivation which comprises administering to a subject a consumer product according to claim 8 that includes a saliva increasing amount of the sensate composition therein.

13. The method of claim 12, wherein the sensate composition is present in the product in an amount of 0.01 to 10% by weight.

14. A method of increasing salivation which comprises administering to a subject a saliva increasing amount of the sensate composition of claim 1.

15. The method of claim 14, wherein the sensate composition is administered in an amount of 0.01 to 10% by weight.
